(19) United States
(12) Patent Application Publication
(10) Pub. No.: US 2006/0025613 A1
(43) Pub. Date: Feb. 2, 2006

(54) Sugar derivatives comprising oxiranes or alpha, beta-unsaturated gamma-lactones, process for their preparation and their utilisation as pesticides

(75) Inventors: Jorge Alberto Guerra Justino, Santarem (PT); Amelia Pilar Grases Santos Silva Rauter, Lisboa (PT); Tana Lukeba Canda, Luanda (AO); Richard Wilkins, Tyne and Wear (GB)

Correspondence Address: MERCHANT & GOULD PC, P.O. BOX 2903, MINNEAPOLIS, MN, 55402-0903, US

(73) Assignees: INSTITUTO POLITECNICO DE SANTAREM/ESCOLA SUPERIOR AGRARIA, Santarem (PT); FACULDADE DE CIENCIAS DA UNIVERSIDADE DE LISBOA, Lisbon (PT); UNIVERSITY OF NEWCASTLE, Newcastle Upon Tyne (GB)

(21) Appl. No.: 10/902,970

(22) Filed: Jul. 30, 2004

(51) Int. Cl. C07D 317/44

(52) U.S. Cl. 549/296

(57) ABSTRACT

The present invention relates to pesticidal compounds with general formula (I), their preparation and application to said pests or their locus, 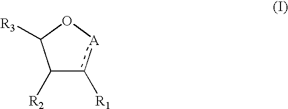
wherein [symbol] represents C—C single or double bond; [symbol] represents —CH(R₄)—, if said C—C is a single bond, R₄ hydrogen, alkoxy or substituted alkoxy, or R₁, R₄, taken together, form an alkylidenedioxy or substituted alkylidenedioxy group; or [symbol] represents ═C(R₅)—C(═O)—, if said C—C is a double bond, R₅ hydrogen, halogen; R₁, R₂-hydrogen, halogen, alkoxy, substituted alkoxy or ester group, or R₁, R₂, taken together with the carbon atoms to which they are attached, represent an oxirane ring; or R₁, R₂, taken together, represent an alkylidenedioxy or substituted alkylidenedioxy group; R₃ represents —CH₂R₆ (R₆-ester group), oxiranyl, or a group of formula 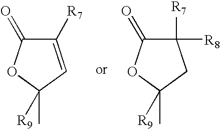
wherein R₇ is hydrogen or alkyl, R₈ phenylsulfanyl, phenylselenyl, phenylsulfoxy or phenylselenoxy; and R₉ hydrogen, ethoxycarbonyl or carbamoyl.

### FIELD OF THE INVENTION

The present invention relates to sugar derivatives comprising oxiranes or α,β-unsaturated γ-lactones with pesticidal, particularly insecticidal, activity, to processes for their preparation and their utilisation as pesticides, which are particularly effective against fruit fly, domestic fly and white fly.

### BACKGROUND OF THE INVENTION

Compounds with the α,β-unsaturated γ-lactone moiety in their structure occur in the plant kingdom, as metabolites of lichens and fungi,¹ as sesquiterpene derivatives² or as steroid glycosides.³

Natural products possessing this structural element are also components of animal species such as sponges.⁴

Many of these compounds exhibit a variety of biologic activities such as antifungal, insecticidal, antibacterial, phytotoxic and anti-inflammatory. Some of them are antibiotics, potential anticancer agents and cyclooxygenase or phospholipase A₂ inhibitors.⁵

Due to their biological importance, several synthetic methods have been developed for the preparation of α,β-unsaturated γ-lactones. The synthesis of the endocyclic lactones (α,β-butenolides) is reported in the literature, and includes mercuration-carbonylation of propargylic alcohols,⁶ condensation of 2,5-bis(trimethylsiloxy)furans with carbonyl compounds in the presence of titanium tetrachloride⁷ and various transformations of C₃ synthons, such as, for example, glycidaldehyde.⁸

Other references report methods for the synthesis of γ-alkylidene-α,β-butenolides⁹ and for the preparation of α,β-butenolide derivatives with insect antifeedant activity.¹⁰

A method for the preparation of the exocyclic type lactones involves the reaction of 2-(bromomethyl)acrylic acid in the presence of indium with carbonyl compounds, to give α-methylene-γ-butyrolactones in 7-96% yield.¹¹

Previous work reports the synthesis of butenolides through the condensation of sugar epoxides with the dianion of phenylselenoacetic acid, followed by hydrolysis and subsequent oxidation of the intermediate phenylselenolactone.^{12, 13, 14} The nucleophilic opening of the oxirane is stereospecific, the configuration of the stereogenic centre in the final lactone being determined by the centre of chirality of the starting epoxide.

Another method for the synthesis of α,β-unsaturated γ-lactones involves a Reformatsky type reaction of a ketosugar or a dialdofuranose with ethyl bromomethylacrylate and zinc in THF under reflux. ^{13, 14, 15}

Ethyl bromomethylacrylate and zinc-silver/graphite at −78° C. have been successfully applied to the synthesis of hydroxyesters from cyclic ketones,¹⁶ ketosugars and a 2,3-O-isopropylidene-D-erythronolactone¹⁷ and to the synthesis of α,β-unsaturated γ-lactones from some ketosugars.¹⁶

The synthesis of 3-ulosonic acids via a samarium iodide Reformatsky reaction of aldonolactones was also reported.¹⁸

Some of this type of compounds, reported in the literature, have fungicidal efficacy.¹³

Epoxy sugars are versatile intermediates in organic synthesis, due to the ease of their preparation from a variety of starting materials and due to their susceptibility to reactions for example with electrophiles, nucleophiles, acids and bases. Furthermore, epoxides are part of a range of compounds recognised as active principles, with biological and pharmacological activity.¹⁹ Reference can be made for example to cytotoxic metabolites, namely crotepoxide, pipoxide and senepoxide, the latter playing an important role in plants as an antiparasitic agent.²⁰

Methods for the preparation of epoxysugars use halohydrins as intermediate compounds,²¹ and also aminosugars,²² tosylates and/or mesylates,²³ vicinal diols,^{24, 25} glycals and carbonyl compounds.²⁷

### SUMMARY OF THE INVENTION

This invention is related to the synthesis and pesticidal utilisation of compounds of general formula (I) described further on.

These compounds possess efficacy as insecticides with high toxicity to fruit fly (Drosophila melanogaster), house fly (Musca domestica) and white fly (Trialeurodes vaporarium).

On the other hand the compounds are not toxic to brine shrimps (Artemia salina), the reference organisms in assays to evaluate the potential toxicity hazard to organisms in ecosystems.

### DETAILED DESCRIPTION OF THE INVENTION

The first object of the invention is a family of compounds with pesticidal activity, of general formula (I): 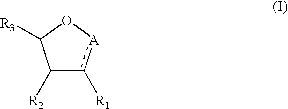
wherein—
[symbol] represents a carbon-carbon single or double bond;
[symbol] represents —CH(R₄)—, if said carbon-carbon bond is a single bond,
   wherein
      R₄ represents, independently, hydrogen, alkoxy or substituted alkoxy, or
      R₁ and R₄, taken together, represent an alkylidenedioxy or substituted alkylidenedioxy group; or
[symbol] represents ═C(R₅)—C(═O)—, if said carbon-carbon bond is a double bond,
   wherein:
      R₅ represents hydrogen or halogen;
R₁ and R₂ represent, independently, hydrogen, halogen, alkoxy, substituted alkoxy or an ester group; or
R₁ and R₂, together with the carbon atoms to which they are attached, represent an oxirane ring; or
R₁ and R₂, taken together, represent an akylidenedioxy or substituted alkylidenedioxy group; and
R₃ represents —CH₂R₆,
   wherein
      R₆ represents an ester group,
oxiranyl,
or a group of formula 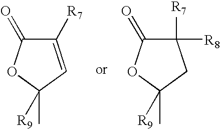
   wherein
      R₇ represents hydrogen or alkyl,
      R₈ represents phenylsulfanyl, phenylselenyl, phenylsulfoxy or phenylselenoxy, and
      R₉ represents hydrogen, ethoxycarbonyl or carbamoyl.

In a first embodiment, the invention relates to a subgroup of compounds within the family of compounds of formula (I), wherein
[symbol] represents a carbon-carbon single bond;
[symbol] represents CH(R₄)—,
   wherein
      R₄ represents, independently, hydrogen, alkoxy or substituted alkoxy, or
      R₁ and R₄, taken together, represent an alkylidenedioxy or a substituted alkylidenedioxy group;
R₁ and R₂ represent, independently, hydrogen, alkoxy, substituted alkoxy or an ester group, or
R₁ and R₂, together with the carbon atoms to which they are attached, represent an oxirane ring; or
R₁ and R₂, taken together, represent an alkylidenedioxy or substituted alkylidenedioxy group; and
R₃ represents a group of formula 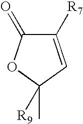
   wherein
      R₇ represents hydrogen or alkyl, and
      R₉ represents hydrogen, ethoxycarbonyl or carbamoyl.

Preferred within this subgroup are the compounds of general formula (IA): 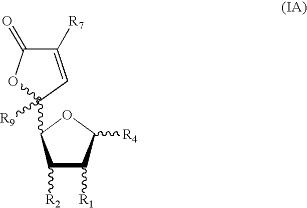
wherein R₁, R₂, R₄, R₇ and R₉ have the meanings indicated for this first embodiment.

Especially preferred are the compounds of formula (IA), wherein R₁ and R₄, taken together, represent the isopropylidenedioxy group, R₂ represents benzyloxy, R₇ represents methyl and R₉ represents hydrogen.

Among these, the most especially preferred are the D-gluco derivative, i.e. 3-O-benzyl-6,7-dideoxy-1,2-O-isopropylidene-7-methyl-α-D-gluco-oct-6-enefuranurono-8,5-lactone or the D-alo derivative, i.e. 3-O-benzyl-6,7-dideoxy-1,2-O-isopropylidene-7-methyl-α-D-alo-oct-6-enefuranurono-8,5-lactone.

Also particularly preferred are compounds of formula (IA), wherein R₁ and R₄, taken together, represent the isopropylidenedioxy group, R₂ represents benzyloxy and R₇ and R₉ represent hydrogen.

Among these, the most especially preferred are the D-alo derivative, i.e. 3-O-benzyl-6,7-dideoxy-1,2-O-isopropylidene-α-D-alo-oct-6-enefuranurono-8,5-lactone and the D-gluco derivative, i.e. 3-O-benzyl-6,7-dideoxy-1,2-O-isopropylidene-α-D-gluco-oct-6-enefuranurono-8,5-lactone.

In a second embodiment, the invention relates to a subgroup of compounds within the family of compounds of formula (I), wherein
[symbol] represents a carbon-carbon double bond;
[symbol] represents ═C(R₅)—C(═O)—,
   wherein,
      R₅ represents hydrogen or halogen;
R₁ represents hydrogen or halogen; and
R₃ represents —CH₂R₆,
   wherein
      R₆ represents an ester group.

Preferred within this subgroup are the compounds of general formula (IB): 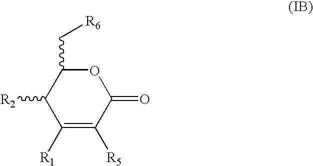
wherein R₁, R₂, R₅ and R₆ have the meanings indicated for this second embodiment.

Especially preferred are the compounds of formula (IB), wherein R₁ represents hydrogen, R₂ represents —OC(═O)CH₃, R₅ represents Br and R₆ represents —OC(═O)CH₃.

Among these, the most especially preferred is the D-erythro derivative, i.e. 4,6-di-O-acetyl-2-bromo-2,3-dideoxy-D-erythro-hex-2-ene-1,5-lactone.

In a third embodiment, the invention relates to a subgroup of compounds within the family of compounds of formula (I), wherein
[symbol] represents a carbon-carbon single bond;
[symbol] represents —CH(R₄)—,
   wherein
      R₄ represents, independently, hydrogen, alkoxy or substituted alkoxy, or
      R₁ and R₄, taken together, represent an alkylidenedioxy or substituted alkylidenedioxy group;
R₁ and R₂ represent, independently, hydrogen, alkoxy or substituted alkoxy, or
R₁ and R₂, together with the carbon atoms to which they are attached, represent an oxirane ring; or
R₁ and R₂, taken together, represent an alkylidenedioxy or a substituted alkylidenedioxy group; and
R₃ represents a group of formula 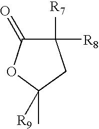
   wherein
      R₇ represents hydrogen or alkyl,
      R₈ represents phenylsulfanyl, phenylselenyl, phenylsulfoxy or phenylselenoxy, and
      R₉ represents hydrogen, ethoxycarbonyl or carbamoyl.

Preferred within this subgroup are the compounds of general formula (IC): 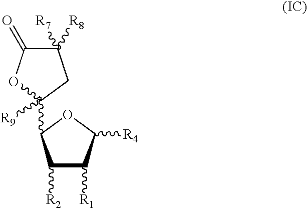
wherein R₁, R₂, R₄, R₇, R₈ and R₉ have the meanings indicated for this third embodiment.

Especially preferred are the compounds of formula (IC), wherein R₁ and R₂, together with the carbon atoms to which they are attached, form an oxirane ring, R₄ represents methoxy, R₇ represents methyl, R₈ represents phenylselenyl and R₉ represents hydrogen.

Among these, the most especially preferred is the 2,3-anhydro-β-L-gulo derivative, i.e. methyl (7R)- and methyl (7S)-2,3-anhydro-6,7-dideoxy-7-methyl-7-phenylselenyl-β-L-gulo-octofuranurono-8,5-lactone.

Also especially preferred are compounds of formula (IC), wherein R₁ and R₄, taken together, form an isopropylidenedioxy group, R₂ represents hydrogen, R₇ represents methyl, R₈ represents phenylselenyl and R₉ represents hydrogen.

Among these, the most especially preferred is the D-ribo derivative, i.e. (7R)- and (7S)-3,6,7-trideoxy-1,2-O-isopropylidene-7-methyl-7-phenylselenyl-α-D-ribo-octofuranurono-8,5-lactone.

Especially preferred are also the compounds of formula (IC), wherein R₁ and R₄, taken together, form an isopropylidenedioxy group, R₂ represents benzyloxy, R₇ represents methyl, R₈ represents phenylselenyl and R₉ represents hydrogen.

Among these, the most especially preferred is the D-gluco derivative, i.e. (7R)- and (7S)-3-O-benzyl-6,7-dideoxy-1,2-O-isopropylidene-7-methyl-7-phenylselenyl-α-D-gluco-octofuranurono-8,5-lactone.

In a fourth embodiment, the invention relates to a subgroup of compounds, within the family of compounds of formula (I), wherein
[symbol] represents a carbon-carbon single bond;
[symbol] represents —CH(R₄)—,
   wherein
      R₄ represents alkoxy or substituted alkoxy;
R₁ and R₂, taken together with the carbon atoms to which they are attached, represent an oxirane ring; and
R₃ represents oxiranyl.

Preferred within this group are the compounds of general formula (ID): 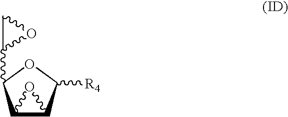
wherein R₄ has the meaning indicated for this fourth embodiment.

Especially preferred are compounds of formula (ID), wherein R₄ represents methoxy.

Among these, the most preferred is the 2,3; 5,6-dianhydro-β-L-gulo derivative, i.e. methyl 2,3; 5,6-dianhydro-β-L-gulofuranoside.

In a fifth embodiment, the invention relates to a subgroup of compounds, within the family of compounds of formula (I), wherein
[symbol] represents a carbon-carbon single bond;
[symbol] represents —CH(R₄)—,
   wherein
      R₄ represents, independently, hydrogen, alkoxy or substituted alkoxy, or
      R₁ and R₄, taken together, represent an alkylidenedioxy or substituted alkylidenedioxy group; or
R₁ and R₂ represent, independently, hydrogen, alkoxy or substituted alkoxy, or
R₁ and R₂, taken together, represent an alkylidenedioxy or substituted alkylidenedioxy group; and
R₃ represents oxiranyl.

Preferred within this subgroup are compounds of general formula (IE): 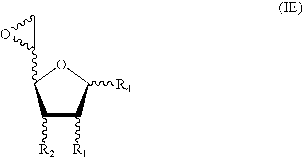
wherein R₁, R₂ and R₄ have the meanings indicated for the fifth embodiment.

A second object of the present invention is a process for the preparation of compounds of formula (I).

### Synthesis of Compounds of Formula (IA)

Compounds of formula (IA) can be prepared by oxidation of phenylsulfanyllactones with m-chloroperbenzoic acid or with sodium metaperiodate, leading to the formation of sulfoxides which, upon pyrolysis in toluene at reflux, provide the corresponding butenolides. The transformation of the phenylselenyllactones into butenolides results from the oxidation with hydrogen peroxide, under acid catalysis. This synthesis is summarized in the following Scheme 1.

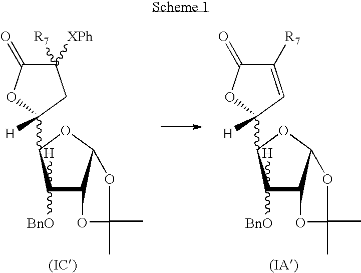

In Scheme 1, a lactone of formula (IC′) [compound of formula (IC), wherein R₁ and R₄ represent, taken together, isopropylidenedioxy, R₂ represents benzyloxy, R₇ represents hydrogen or methyl, R₈ represents XPh, wherein X represents S or Se, and R₉ represents hydrogen] is converted into a butenolide of formula (IA′) [compound of formula (IA), wherein R₁ and R₄ represent, taken together, isopropylidenedioxy, R₂ represents benzyloxy, R₇ represents hydrogen or methyl and R₉ represents hydrogen]. This conversion is carried out in the presence of m-chloroperbenzoic acid (when X represents S) and toluene at reflux, or in the presence of hydrogen peroxide, in acid medium (when X represents Se), at temperatures between −20° C. and room temperature.

### Synthesis of Compounds of Formula (IB)

Compounds of formula (IB) can be prepared according to Scheme 2.

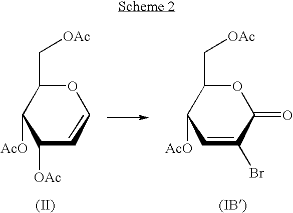

In Scheme 2, a compound of formula (II) is reacted with N-bromosuccinimide in the presence of tetrahydrofuran and water, at a temperature of 10° C. to 50° C., for a period of time from 4 to 24 hours. In a second step the product obtained is added to molecular sieves and pyridinium chlorochromate in dichloromethane, at a temperature of 10° C. to 50° C., for a period of time from 8 to 24 hours, to yield an α,β-unsaturated hexono-1,5-lactone of formula (IB′) [compound of formula (IB), wherein R₁ represents hydrogen, R₂ and R₆ represent acetoxy and R₅ represents bromo].

### Synthesis of Compounds of Formula (IC)

Compounds of formula (IC) can be prepared by reaction of the corresponding epoxide precursor with dianions, namely: dianion of phenylselenoacetic acid, phenylselenopropionic acid and phenylthioacetic acid, which upon cyclization in acid medium yield the corresponding phenylselenyllactones or phenylsulfanyllactones, according to Schemes 3a and 3b.

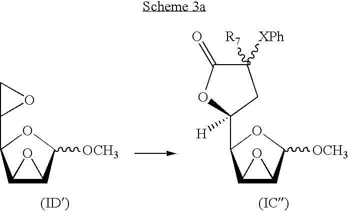

In Scheme 3a, a diepoxide of formula (ID′) [compound of formula (ID), wherein R₁ and R₂ represent, taken together, oxiranyl and R₄ represents methoxy] is converted into a lactone of formula (IC″) [compound of formula (IC), wherein R₁ and R₂ represent, taken together, oxiranyl, R₄ represents methoxy, R₇ represents hydrogen or methyl, R₈ represents XPh, wherein X represents S or Se, and R₉ represents hydrogen]. This conversion is carried out by treatment of (ID′) with lithium diisopropropylamide in tetrahydrofuran, at a temperature between −10° C. and 10° C., followed by reaction with a compound of formula PhXCHR₇CO₂H (wherein X represents S or Se e R₇ represents hydrogen or methyl).

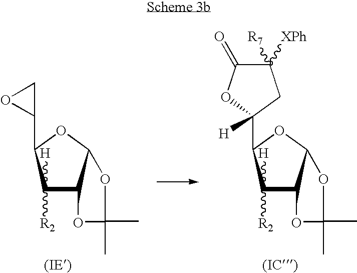

In Scheme 3b, following a procedure similar to that of Scheme 3a, an epoxide of formula (IE′) [compound of formula (IE), wherein R₁ and R₄ represent, taken together, isopropylidenedioxy and R₂ represents benzyloxy or hydrogen] is converted into a lactone of formula (IC′″) [compound of formula (IC), wherein R₁ and R₄ represent, taken together, isopropylidenedioxy, R₂ represents benzyloxy or hydrogen, R₇ represents hydrogen or methyl, R₈ represents XPh, wherein X represents S or Se, and R₉ represents hydrogen].

### Synthesis of Compounds of Formula (ID)

Compounds of formula (ID) can be prepared according to Scheme 4.

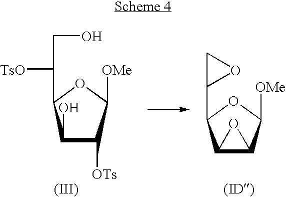

In Scheme 4, a compound of formula (III) is converted into a diepoxide of formula (ID″) [compound of formula (ID), wherein R₄ represents methoxy]. This conversion is carried out by treatment of a compound of formula (III) with aqueous solution of potassium hydroxide and tetrahydrofuran, at a temperature between 5° C. and 40° C.

### Synthesis of Compounds of Formula (IE)

Compounds of formula (IE) can be prepared according to Scheme 5.

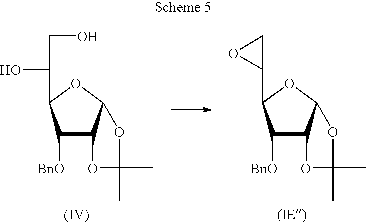

In Scheme 5, a compound of formula (IV) is converted into an epoxide of formula (IE″) [compound of formula (IE), wherein R₁ and R₄ represent, taken together, isopropylidenedioxy and R₂ represents benzyloxy]. This conversion is carried out by conversion of compound of formula (IV) with triphenylphosphane in benzene, followed by the addition of molecular sieves and diethyl azodicarboxylate, at a temperature between 60° C. and 100° C., for a period of time of 1 to 4 days.

A third object of the instant invention is the use of compounds of formula (I) as pesticides.

Preferably, these compounds are used as arthropodicides.

More preferably, these compounds are used as insecticides.

Still more preferably, these compounds are used with particular efficacy as insecticides of high toxicity for controlling fruit fly (Drosophila melanogaster), house fly (Musca domestica) and white fly (Trialeurodes vaporarium).

A fourth object of the invention is a method for controlling pests, namely arthropods, particularly insects, especially fruit fly, house fly and white fly. Said method comprises the application of an effective amount of compounds of formula (I) to said pests or their locus.

### EXPERIMENTAL

### Preparation Examples

### EXAMPLE 1

### Preparation of 3-O-benzyl-6,7-dideoxy-1,2-O-isopropylidene-7-methyl-α-D-gluco-oct-6-enefuranurono-8,5-lactone (1)

Glacial acetic acid (1 drop) was added to a solution of (7R)- and (7S)-3-O-benzyl-6,7-dideoxy-1,2-O-isopropylidene-7-methyl-7-phenylselenyl-α-D-gluco-octofuranurono-8,5-lactone (130 mg, 0.26 mmol) in anhydrous tetrahydrofuran (0.26 mL) at 0° C. 30% H₂O₂ (6.70 eq.) was added dropwise and the reaction mixture was stirred for 45 min at 0° C. Extraction with a saturated solution of NaHCO₃ was followed by extraction with CH₂Cl₂ (3×2 mL). The organic phase was dried over sodium sulphate and evaporated under vacuum. The residue was purified by low pressure column chromatography to provide the title compound (45 mg, 64%) and unreacted starting material (26 mg, 20%); R_{f}: 0.40 (ethyl acetate/n-hexane 1:4); [α]_{D}²⁰=−22 (c 1.0; CHCl₃); IR (neat): 1770 (C═O), 1378 (C—O, isopropyl), 1662 (C═C) cm⁻¹; ¹H NMR (300 MHz, CDCl₃): δ 7.35-7.30 (m, 6H, H-6, Ph), 5.93 (d, 1H, H-1, J_{1,2}=3.6 Hz), 5.17 (dd, 1H, H-5, J_{4,5}=8.6 Hz, J_{5,6}=1.5 Hz), 4.69 (s, 2H, OCH₂Ph), 4.63 (d, 1H, H-2), 4.14 (d, 1H, H-3, J_{3,4}=3.0 Hz), 3.91 (dd, 1H, H-4), 1.93 (s, 3H, Me-7), 1.48 (s, 3H, Me, isopropyl), 1,25 (s, 3H, Me, isopropyl); ¹³C NMR (75.43 MHz, CDCl₃): δ 174.0 (C═O), 148.7 (C-6), 137.1 (Cq, Ph), 130.2 (C-7), 128.5; 128.1; 127.8 (Ph), 112.3 (Cq, isopropyl), 105.3 (C-1), 82.3 (C-2), 81.8 (C-3), 81.5 (C-4), 76.8 (C-5), 72.8 (OCH₂Ph), 10.7 (Me-7), 26.73 (Me, isopropyl), 26.11 (Me, isopropyl). Anal. calcd for C₁₉H₂₂O₆ (346.35): C, 65.89; H, 6.39; Found: C, 65.57; H, 6.45%.

### EXAMPLE 2

### Preparation of 3-O-benzyl-6,7-dideoxy-1,2-O-isopropylidene-7-methyl-α-D-alo-oct-6-enefuranurono-8,5-lactone (2)

Following a procedure similar to that of Example 1, starting from (7R)- and (7S)-3-O-benzyl-6,7-dideoxy-1,2-O-isopropylidene-7-methyl-7-phenylselenyl-α-D-gluco-octofuranurono-8,5-lactone¹⁵ (100 mg, 0.2 mmol), the title compound was obtained (40 mg, 73% taking into account the starting material that reacted), with recovery of unreacted starting material (20 mg, 20%); R_{f}: 0.23 (ethyl acetate/n-hexane 1:3); [α]_{D}²⁰=+122 (c 1.0; CHCl₃); IR (neat): 1786 (C═O), 1384 (C—O, isopropyl), 1662 (C═C) cm⁻¹; ¹H NMR (300 MHz, CDCl₃): δ 7.41-7.30 (m, 5H, Ph), 7.25 (s, 1H, H-6), 5.77 (d, 1H, H-1, J_{1,2}=3.6 Hz), 5.16 (br s, 1H, H-5), 4.68; 4.64 (part A of AB system, OCH₂Ph, J_{AB}=12 Hz), 4.56 (t, 1H, H-2, J_{2,3}=4.4 Hz), 4.49; 4.45 (part B of AB system, 4.32 (dd, 1H, H-4, J_{3,4}=8.7 Hz, J_{4,5}=3.0 Hz), 3.65 (dd, 1H, H-3), 1.93 (s, 3H, Me-7), 1.55 (s, 3H, Me, isopropyl), 1.39 (s, 3H, Me, isopropyl); ¹³C NMR (75.43 MHz, CDCl₃): δ 173.8 (C═O), 145.3 (C-6), 136.9 (Cq, Ph), 130.4 (C-7), 128.5; 128.2; 128.0 (Ph), 113.3 (Cq, isopropyl), 104.1 (C-1), 79.3 (C-5), 78.6 (C-4), 72.1 (OCH₂Ph), 76.6 (C-2), 75.5 (C-3), 26.8 (Me, isopropyl), 26.5 (Me, isopropyl), 10.7 (Me-7). Anal. calcd for C₁₉H₂₂O₆ (346.35): C, 65.89; H, 6.39; Found: C, 65.63; H, 6.48%.

### EXAMPLE 3

### Preparation of 3-O-benzyl-6,7-dideoxy-1,2-O-isopropylidene-α-D-alo-oct-6-enefuranurono-8,5-lactone (3)

Following a procedure similar to that of Example 1, starting from (7R)- and (7S)-3-O-benzyl-6,7-dideoxy-1,2-O-isopropylidene-7-phenylselenyl-α-D-alo-octofuranurono-8,5-lactone¹⁵ (130 mg, 0.3 mmol) the title compound was obtained (100 mg, 91%). R_{f} 0,60 (ethyl acetate/n-hexane 1:1); [α]_{D}²⁰=+47 (c 0.5; CH₂Cl₂); IR (KBr): 1774 (C═O), 1382 (C—O, isopropyl), 1662 (C═C) cm⁻¹; ¹H NMR (300 MHz, CDCl₃): δ 7.42 (d, H-6, J_{6,7}=6 Hz), 7.36-7.29 (m, 5H, Ph), 6.08 (dd, H-7, J_{5,7}=2.1 Hz), 5.7 (d, H-1, J_{1,2}=3.6 Hz), 5.28 (br s, H-5), 4.65; 4.61, 4.47 and 4.43 (AB system, OCH₂Ph, J_{AB}=12 Hz), 4.51 (dd, H-2, J_{2,3}=4.2 Hz), 4.33 (dd, H-4, J_{4,5}=3.3 Hz, J_{3,4}=9 Hz), 3.62 (dd, H-3), 1.59 (s, 3H, Me, isopropyl), 1.35 (s, 3H, Me, isopropyl); ¹³C NMR (75,43 MHz, CDCl₃): δ 173.8 (C═O), 152.9 (C-6), 136.4 (Cq, Ph), 128.6; 128.3 (Ph), 121.9 (C-7), 112.0 (Cq, isopropyl), 104.3 (C-1), 86.8 (C-5), 81.5 (C-4), 78.5 (C-2), 72.2 (OCH₂Ph), 68.0 (C-3), 26.5 (Me, isopropyl), 26.3 (Me, isopropyl). Anal. calcd for C₁₈H₂₀O₆ (332.33): C, 65.06; H, 6.06; Found: C, 65.05; H, 6.29%.

### EXAMPLE 4

### Preparation of 3-O-benzyl-6,7-dideoxy-1,2-O-isopropylidene-α-D-gluco-oct-6-enefuranurono-8,5-lactone (4)

Following a procedure similar to that of Example 1, starting from (7R)- and (7S)-3-O-benzyl-6,7-dideoxy-1,2-O-isopropylidene-7-phenylselenyl-α-D-gluco-octofuranurono-8,5-lactone ^{13,15} (112 mg, 0.23 mmol) the title compound was obtained (57,4 mg, 75%). R_{f} 0.32 (ethyl acetate/n-hexane 1:4); m.p.=66-70° C.; [α]_{D}²⁰=+0.2 (c 0.5; CHCl₃); IR (CHCl₃): 1758 (C═O), 1380 (C—O, isopropyl) cm⁻¹; ¹H NMR (300 MHz, CDCl₃): δ 7.71 (dd, H-6, J_{6,7}=5.9 Hz), 7.37-7.31 (m, 5H, Ph), 6.15 (dd, H-7, J_{5,7}=1.5 Hz), 5.92 (d, H-1, J_{1,2}=3.5 Hz), 5.30 (dd, H-5, J_{5,6}=1.5 Hz), 4.73-4.65 (AB system, OCH₂Ph, J_{AB}=11 Hz), 4.63 (d, 1H, H-2), 4.15 (d, H-3, J_{3,4}=3.5 Hz), 3.97 (dd, H-4, J_{4,5}=6.7 Hz), 1.29 (s, 3H, Me, isopropyl), 1.43 (s, 3H, Me, isopropyl); ¹³C NMR (75.43 MHz, CDCl₃): δ 172.7 (C═O), 156.5 (C-6), 137.0 (Cq, Ph), 128.6; 128.3; 128.1 (Ph), 121.7 (C-7), 112.3 (Cq, isopropyl), 105.4 (C-1), 82.3 (C-2), 81.8 (C-3), 81.2 (C-4), 79.2 (C-5), 72.9 (OCH₂Ph), 26.8 (Me, isopropyl), 26.2 (Me, isopropyl). Anal. calcd for C₁₈H₂₀O₆ (332.33): C, 65.06; H, 6.06; Found: C, 64.79; H, 6.20%.

### EXAMPLE 5

### Preparation of compound 4,6-di-O-acetyl-2-bromo-2,3-dideoxy-D-erythro-hex-2-enone-1,5-lactone (5)

N-bromosuccinimide (780 mg, 4.88 mmol) was added to a solution of tri-O-acetyl-D-glucal (1 g, 3.67 mmol) in 370 mL tetrahydrofuran (THF) and 92 mL distilled water. The mixture was stirred overnight at room temperature. The reaction mixture was poured into water chilled at 0° C., followed by extraction of the mixture thus obtained with diethyl ether. The combined organic phases were dried over sodium sulphate, filtered and the solvent was removed at reduced pressure to afford a syrup. The residue was then added to a suspension of 3 Å molecular sieves (6.8 g) and pyridinium chlorochromate (4.4 g) in dichloromethane (20 mL). The reaction mixture was stirred at room temperature overnight, eluted with diethyl ether and stirred at room temperature for 10 min. The precipitate was removed by filtration and the filtrate was poured over florisil in a filter and filtered under vacuum. The colourless filtrate was concentrated under reduced pressure to provide the title compound as a syrup (760 mg, 37%). [α]_{D}²⁰=+121 (c 1, CH₂Cl₂); IR (neat): 1752 cm⁻¹ (C═O, lactone); 1632 cm⁻¹ (C═C); ¹H NMR (300 MHz, CDCl₃): δ 7.01 (d, 1H, H-3, J_{3,4}=3.9 Hz); 5.30 (dd, 1H, H-4, J_{4,5}=6.6 Hz); 4.19, 4.17, 4.15, 4.13 (H-6a, part A of AB system, J_{6a,6b}=12.6, J_{5,6a}=4.5 Hz); 4.10, 4.08, 4.06, 4.04 (H-6b, part B of AB system, J_{5,6}b=3.6 Hz); 1.91 (s, 3H, acetyl CH₃); 1.85 (s, 3H, acetyl CH₃); ¹³C NMR (75.43 MHz, CDCl₃): δ 170.1, 169.0 (acetyl C═O); 157.1 (lactone C═O); 142.3 (C-3); 116.4 (C-2); 77.7 (C-4); 64.8 (C-5); 61.6 (C-6), 20.4 (acetyl CH₃); Anal. calcd for C₁₀H₁₁O₆Br (307.08): C, 39.12; H, 3.61; Found: C, 39.13; H 3.62%.

### EXAMPLE 6

### Preparation of methyl (7R)-/(7S)-2,3-anhydro-6,7-dideoxy-7-methyl-7-phenylselenyl-α-L-gulo-octofuranurono-8,5-lactone (6A/6B)

A solution of n-BuLi (1,6 M in n-hexane, 5.43 mL, 8.7 mmol) was added dropwise to a solution of diisopropylamine (1.22 mL, 8.7 mmol) in anhydrous tetrahydrofuran (16 mL) under argon atmosphere at 0° C., and the mixture was stirred at 0° C. for 25 min. A solution of phenylselenoacetic, phenylselenopropionic or phenylthioacetic acid (3.9 mmol) in anhydrous tetrahydrofuran (4 mL) was added dropwise to the reaction mixture, keeping the temperature at 0° C. and the mixture was stirred for 1 h at 0° C. A solution of methyl 2,3; 5,6-dianhydro-β-L-gulofuranoside (compound of Example 9, 624 mg, 3.95 mmol) in anhydrous tetrahydrofuran (3 mL/g of compound of Example 9) was then added dropwise and the reaction mixture was stirred first at 0° C. for 1 h and then at room temperature for 16 h. After addition of a solution of 50% acetic acid (10 mL) and heating under reflux for six hours, the mixture was cooled to room temperature and neutralised with a saturated solution of NaHCO₃. After extraction of the mixture with diethyl ether (3×20 mL), the combined organic phases were washed with water and dried over sodium sulphate. Evaporation of the solvent under vacuum and purification by low pressure column chromatography, gave the title compounds (460 mg, 32%, 6A/6B: 3/1). R_{f}=0.23 (ethyl acetate/n-hexane 1:1); IR (neat): 1784 (C═O), 1286 (C—O, epoxide) cm^{-1;} ¹H NMR (300 MHz, CDCl₃) of 6A: δ 7.68-7.65 (m, 2H, Ph), 7.48-7.36 (m, 3H, Ph), 5.08 (s, 1H, H-1), 4.77 (ddd, 1H, H-5, J_{4,5}=6,9 Hz, J_{5,6a}=5.7 Hz, J_{5,6}=10.5 Hz), 4.02 (d, 1H, H-4, J₃₄=6.6 Hz), 3.74 (d, 1H, H-3, J_{2,3}=2.7 Hz), 3.68 (d, 1H, H-2), 3.55 (s, 3H, OCH₃), 2.52 (dd, 1H, H-6a, J_{6a,6b}=14.1 Hz), 2.32 (dd, 1H, H-6b), 1.66 (s, 3H, Me); ¹³C NMR (75.43 MHz, CDCl₃) of 6B: δ 176.0 (C-8), 137.7 (Cq, Ph), 129.7; 128.9 (Ph), 102.2 (C-1), 77.0 (C-4), 75.7 (C-5), 56.8 (OMe), 54.7 (C-2), 53.4 (C-3), 44.5 (C-7), 39.0 (C-6), 23.9 (Me). Anal. calcd for C₁₆H₁₈O₅Se (369.25): C, 52.04; H, 4.90; Found: C, 51.78; H, 4.95%.

### EXAMPLE 7

### Preparation of (7R)-/(7S)-3,6,7-Trideoxy-1,2-O-isopropylidene-7-methyl-7-phenylselenyl-α-D-ribo-octofuranurono-8,5-lactone (7A/7B)

Following a procedure similar to that of Example 6, starting from 5,6-anhydro-3-deoxy-1,2-O-isopropylidene-α-D-ribo-hexofuranose¹⁵ (380 mg, 2.04 mmol), the title compounds were obtained (470 mg, 58%, 7A/7B: 7/3), after separation by column chromatography with ethyl acetate/n-hexane (1:3) as eluent; R_{f}=0.27 (ethyl acetate/n-hexane (1:3); IR (KBr): 1754 (C═O), 1378 (C—O, isopropyl) cm⁻¹; ¹H NMR (300 MHz, CDCl₃) of 7A: δ 7.74-7.67 (m, 2H, Ph), 7.49-7.29 (m, 3H, Ph), 5.84 (d, 1H, H-1, J_{3,2}=3.3 Hz), 4.78 (t, 1H, H-2, J_{2,3}b=3.9 Hz), 4.48-4.41 (ddd, 1H, H-5, J_{4,5}=5.1 Hz, J_{5,6b}=10.1 Hz), 4.31-4.24 (ddd, 1H, H-4), 2.52 (dd, 1H, H-6a, J_{5,6a}=5.5 Hz, J_{6a,6b}=14.1 Hz), 2.27-2.14 (m, 2H, H-6b, H-3a), 1.73-1.66 (m, 4H, H-3b, Me), 1.53 (s, 3H, Me), 1.35 (s, 3H, Me). ¹³C NMR (75.43 MHz, CDCl₃) of 7B: δ 176.4 (C═O), 137.8 (Cq, Ph), 129.9; 129.1 (Ph), 111.6 (Cq, isopropyl), 105.6 (C-1), 80.2 (C-2), 78.6 (C-4), 76.9 (C-5), 44.7 (C-7), 40.5 (C-6), 35.1 (C-3), 26.7 (Me), 26.1 (Me), 24.0 (Me). Anal. calcd for C₁₈H₂₂O₅Se (397.30): C, 54.41; H, 5.57; Found: C, 54.79; H, 5.72%.

### EXAMPLE 8

### Preparation of (7R)-/(7S)-3-O-Benzyl-6,7-dideoxy-1,2-O-isopropylidene-7-methyl-7-phenylselenyl-α-D-gluco-octofuranurono-8,5-lactone (8A/8B)

Following a procedure similar to that of Example 6, starting from 5,6-anhydro-3-O-benzyl-1,2-O-isopropylidene-α-D-glucofuranose (430 mg, 1.47 mmol), the title compounds were obtained (590 mg, 79%, 8A/8B:1/2), after separation by chromatography with ethyl acetate/n-hexane (1:6) as eluent; R_{f}=0,49 (ethyl acetate/n-hexane 1:6); IR (neat): 1773 (C═O), 1382 (C-0, isopropyl) cm⁻¹; ¹H NMR (300 MHz, CDCl₃): δ 7.67-7.63 (m, 4H, Ph), 7.41-7.26 (m, 16H, Ph), 5.95 (d, 1H, H-1A, J_{3,2}=3.6 Hz), 5.90 (d, 1H, H-1B, J_{1,2}=3.6 Hz), 4.83-4.17 (m, 7H, OCH₂Ph, H-2, H-5A), 4.18 (dd, 1H, H-4A, J_{3,4}=10.2 Hz, J_{4,5}=3.3 Hz), 4.09-4.05 (m, 2H, H-3), 3.78-3.75 (dd, 1H, H-4B, J_{3,4}=7.2 Hz, J_{4,5}=3.3 Hz), 3.16 (d, 1H, H-5B, J_{5,6}=3.9 Hz), 2.94 (dd, 1H, H-6aB, J_{5,6a}=3.9 Hz, J_{6a, 6b}=5.1 Hz), 2.79 (dd, 1H, H-6bB, J_{5,6b}=2.4 Hz), 2.59 (dd, 1H, H-6aA, J_{5,6a}=5.7 Hz J_{6a,6b}=14.4 Hz), 2.38 (dd, 1H, H-6bA, J_{5,6b}=9.9 Hz), 1.63 (s, 6H, Me-7), 1.51 (s, 6H, Me, isopropyl), 1,31 (s, 6H, Me, isopropyl); ¹³C NMR (75.43 MHz, CDCl₃): δ 176.7 (C═O), 137.6 (CqPh), 129.8; 129.0; 128.5; 128.1; 127.8; 127.6 (Ph), 112.1 (Cq-isopropyl), 105.2 (C-1), 82.6 (C-2), 82.5, 82.0 (C-3), 81.7, 81.5 (C-4), 73.1 (C-5A), 72.6 (OCH₂Ph), 48.2 (C-5B), 46.9 (CH₂-6A), 45.0, 44.2 (C-7), 41.3 (CH₂-6B), 26.8 (Me, isopropyl), 26.2 (Me, isopropyl), 24.0 (Me-7). Anal. calcd for C₂₅H₂₈O₆Se (503.42): C, 59.65; H, 5.60; Found: C, 60.00; H, 5.93%.

### EXAMPLE 9

### Methyl 2,3;5,6-Dianhydro-β-L-gulofuranoside (9)

### a) Preparation of methyl 2,5-di-O-tosyl-β-D-glucofuranoside

A solution of 1-O-methyl-2,5-O-ditosyl-β-D-glucofuranurono-6,3-lactone²⁹ (500 mg, 1 mmol) in tetrahydrofuran (10 mL) was added dropwise over 1 h to a suspension of LiBH₄ (42 mg, 1.93 mmol) in tetrahydrofuran (5 mL), previously cooled to −10° C. The reaction mixture was then stirred at +14° C. for 16 h. After neutralisation with acetic acid (50% in H₂O), filtration and concentration under reduced pressure a syrup was obtained which was treated with MeOH (3×5 mL) and concentrated under vacuum. The residue was dissolved in ethyl acetate and extracted with water. The organic phase was dried over sodium sulphate and evaporated at reduced pressure. The residue was purified by column chromatography with ethyl acetate/toluene (1:3) as eluent to give the title compound (472 mg, 94%). R_{f}: 0.25 (ethyl acetate/toluene 1:3); [α]_{D}²⁰=+44 (c 1, CHCl₃); IR (KBr): 3500 (OH) cm⁻¹; ¹H NMR (300 MHz, CDCl₃): δ 7.85-7.82 (m, 4H, Ph), 7.41-7.37 (m, 4H, Ph), 4.92-4.88 (m, 2H, H-1, H-5), 4.70 (s, 1H, H-2), 4.35 (t, 1H, H-3, J_{3,OH}=8.1 Hz), 4,32 (dd, 1H, H-4, J_{4,5}=3.9 Hz, J_{3,4}=8.4 Hz), 3.90-3.82 (m, 2H, H-6a, H-6b), 3.31 (s, 3H, OCH₃), 2.46 (s, 3H, CH₃Ph), 2.45 (s, 3H, CH₃Ph); ¹³C NMR (75.43 MHz, CDCl₃): δ 145.0; 145.1 (Cq, Ph), 130.0; 129.8; 128.8; 127.9 (CH, Ph), 100.8 (C-1), 83.3 (C-2), 79.2 (C-5), 76.7 (C-4), 73.5 (C-3), 61.7 (C-6), 56.0 (OMe), 21.7 (CH₃, Ts). Anal. calcd for C₂₁H₂₆O₁₀S₂ (502.54): C, 50.21; H, 5.21; S 12.76; Found: C, 50.23; H, 5.30; S 12.65%.

### b) Methyl 2,3;5,6-dianhydro-β-L-gulofuranoside

A solution of KOH (56 mg, 1.0 mmol) in water (1 mL) was cooled to +10° C. and added to a solution of methyl 2,5-di-O-tosyl-α-D-glucofuranoside (compound of step a)) (202 mg, 0.4 mmol) in water (4.0 mL) and tetrahydrofuran (1.0 mL), previously cooled to +10° C. The reaction mixture was stirred at room temperature, until monitoring by thin layer chromatography showed that the reaction was completed. After extraction with CHCl₃ (10×10 mL), the combined organic phases were dried over sodium sulphate and concentrated at reduced pressure. The residue was purified by column chromatography with ethyl acetate/toluene (1:3) to provide the title compound (61 mg, 97%); R_{f}: 0.5 (ethyl acetate/n-hexane 1/1); [α]_{D}²⁰=+66 (c 0.5; CH₂Cl₂); ¹H NMR (300 MHz, CDCl₃): δ 5.01 (s, 1H, H-1), 3.78 (d, 1H, H-4, J_{4,5}=6.0), 3.68-3.65 (m, 2H, H-2, H-3), 3.43 (s, 3H, OCH₃), 3.19 (ddd, 1H, H-5), 2.87 (dd, 1H, H-6a, J_{5,6a}=4.2 Hz), 2.75 (dd, 1H, H-6b, J_{5,6}b=3 Hz, J_{6a,6b}=6 Hz); ¹³C NMR (75.43 MHz, CDCl₃): δ 102.4 (C-1), 76.7 (C-4), 55.7 (OMe), 55.6 (C-2), 53.7 (C-3), 50.4 (C-5), 44.0 (C-6). Anal. calcd for C₇H₁₀O₄ (158.14): C, 53.16; H, 6.36; Found: C, 53.13; H, 6.35%.

### EXAMPLE 10

### Preparation of 5,6-anhydro-3-O-benzyl-1,2-O-isopropylidene-α-D-alofuranose

Triphenylphosphane (2.6 eq.) was added to a solution of 3-O-benzyl-1,2-O-isopropylidene-α-D-alofuranose²⁸ (2.40 g, 8.2 mmol) in benzene (143.5 mL) and the mixture was stirred at room temperature for 15 min. After addition of powdered 3 Å molecular sieves (6.56 g), diethyl azodicarboxylate (2.6 eq.) was added dropwise and the reaction mixture was stirred at 80° C. for 48 h. After filtration and evaporation of the solvent, the residue was purified by low pressure column chromatography to provide the title compound (1.75 g, 73%) as a syrup after purification by column chromatography with the system ethyl acetate/n-hexane (1:3); R_{f}=0.41 (ethyl acetate/n-hexane 1:3); [α]_{D}²⁰ =+62 (c 1.0; CHCl₃); IR (neat): 1262 (C—O, epoxide), 1380 (C—O, isopropyl) cm⁻¹; ¹H NMR (300 MHz, CDCl₃): δ 7.39-7.29 (m, 5H, Ph), 5.74 (d, 1H, H-1, J_{1,2}=3.6 Hz), 4.76; 4.72 (part A of AB system, OCH₂Ph, J_{A,B}=11.7 Hz), 4.59-4.55 (m, 2H, H-2, OCH₂Ph, part B of AB system), 3.66 (dd, 1H, H-3, J_{2,3}=4.2 Hz, J_{3,4}=8.7 Hz), 3.19-3.16 (m, 1H, H-5), 4.20 (dd, 1H, H-4, J_{4,5}=3.0 Hz), 2.79-2.73 (m, 2H, H-6a, H-6b), 1.53 (s, 3H, Me), 1.37 (s, 3H, Me); ¹³C NMR (75.43 MHz, CDCl₃): δ 137.1 (Cq, Ph), 128.5; 128.3; 127.9 (Ph), 112.8 (Cq, isopropyl), 103.8 (C-1), 71.8 (OCH₂Ph), 77.5 (C-2), 77.0 (C-3), 76.5 (C-4), 50.4 (C-5), 44.2 (C-6), 26.6 (Me), 26.4 (Me). Anal. calcd for C₁₆H₂O₅ (292.3): C, 65.74; H, 6.88; Found: C, 65.40; H 6.88%.

### Biological Activity of the Compounds

Materials and Methods for Determination of Biological Activity

A range of arthropod species was chosen to represent those in the terrestrial, aerial and aquatic environment, covering important target pest groups such as house and fruit flies and the white fly, which belongs to a group of agricultural and horticultural pests.

### Method A

### Topical Treatment of Adult Fruit Fly (D. Melanogaster)

A culture of fruit flies (D. melanogaster) was used in the production of adult flies (approx 0.22 mg) of about seven days.

Serial dilutions of the compounds were prepared in acetone, and volumes of approximately 0.2 μL were applied to the ventral surface of each insect, using a calibrated PAX 100 microapplicator and a 1 mL syringe.

The fruit flies, in groups of 5, were anaesthetised using carbon dioxide. Prior to recovery the flies were placed in a containing vial and kept at 30±1° C., for observations of mortality at 1, 2, 3 and 24 hours after treatment.

For this purpose solutions of 6 different concentrations and a control were employed in groups of 12 and 20 insects. Control mortalities were normally zero but occasionally rose to 5-10%.

### Method B

### Second Method for Topical Treatment of Adult Fruit Flies

Different dilutions of the compounds in acetone were prepared and applied to individual fruit flies using a Gilson piston micropipette.

Individual flies were held in padded forceps and 1 μL of acetone solution was applied. The acetone was allowed to evaporate before placing the fly into the vial for observation, kept at 30±1° C., following the standard methodology.

### Method C

### Method by Feeding Adult Fruit Fly (D. melanogaster)

Large glass vials were used which were fitted with caps, inside of which is inserted a piece of cotton wool. This was soaked in a 10% sugar solution containing the test compounds in a given concentration.

Care was taken to ensure that no solution dripped from the cotton wool and condensation was avoided by keeping the vials at room temperature (25° C.).

The fruit flies were anaesthetised using carbon dioxide and placed in the vials for recovery and feeding.

### Method D

### Topical Treatment of Fruit Fly Larvae

Fruit fly larvae were separated from the growing medium and second and third instars were topically treated with 0.2 μL acetone solutions of the compounds using a PAX microapplicator.

Larvae were held in padded forceps for dosing and then placed on moistened filter paper, at 30±1° C., for observation.

### Method E

### Topical Treatment of House Fly Adults (M. domestica)

A culture of an insecticide-susceptible strain, Cooper, was established. Three-day-old adult house flies (body weight about 1.8 mg) were anaesthetised with carbon dioxide. Using the PAX microapplicator, a volume of 1 μL of acetone solution of the test compounds was applied to the dorsal cuticle, holding the fly in padded forceps.

Groups of flies were placed into closed vials, kept at 30±1° C., for observation.

### Method F

### Immersion Bioassay of Brine Shrimp A. salina Larvae in Brine

Freshly hatched brine shrimps were prepared by adding aquarium brine shrimp eggs to salt water (15 g sea salt per litre water). The following day hatchlings were separated from eggs and empty egg cases, using their phototropic movement and a Pasteur pipette.

Into each of a set of small glass vials was pipetted 195 μL of brine containing 5 shrimp larvae, using a micropipette. The solution of the test compound in 5 μL acetone was added, the vial closed and kept at 30±1° C. for observation.

Dead and alive shrimps were counted, at 1 and 24 hours after treatment, using a microscope. Six concentrations and a control were used with 10 shrimps treated.

A blank test was performed for comparison of the results.

### Method G

### Foliar Treatment of Glasshouse White Fly (T. vaporariorum)

Seedlings of tomato plants were infested with adult white fly. Selected leaves of the tomato plants were excised and carefully trimmed to 3 leaflets without disturbing the infestation. These were placed in glass tubes containing water and the leaflets were then sprayed on both sides with a small sprayer delivering for each one 200 μL of a solution of the test compound dissolved in 30% acetone in water.

Controls were sprayed with the solvent alone.

Counts of insects on the individual leaflets were made immediately after spraying and then at 14 hours following.

### Calculation of Toxicity Parameters

Dosages used in insect treatments were based upon the amount of compound applied to each insect. For the shrimps the final concentrations of the compounds in the immersion brine were used.

The 24 hour mortalities were used to calculate the LD₅₀/LC₅₀ using regression analysis of the probability percent mortality (probit) against log dose/concentration.³⁰ This was calculated using PoloPC software (LeOra Software, Berkeley, Calif., 1994).

Where single dose treatments were used (in the case of whitefly assays), no statistics are available and the results are expressed as percent effect.

### Results

### Toxicity to Fruit Fly (D. Melanogaster)

The results of assays with fruit fly D. Melanogaster are given in Table 1.

In the table are also given the confidence intervals at 95% for the LD₅₀ values, the number of organisms tested, the slope obtained by linear regression and the index g (of significance). Data are considered satisfactory if g is substancially less than 1 and seldom greater than 0.4.³¹

As regards the confidence intervals for LD₅₀, the indication (90%) means that the intervals were calculated at 90% and not at 95%.

From the analysis of the LD₅₀ values, calculated through method 1, it is found that in general the compounds tested are active against adult fruit flies, compounds of Examples 1, 2, 3 and 9 being extremely active.

All of them are more active than imidacloprid, the reference insecticide for fruit fly.

TABLE 1
Toxicity parameters of compounds tested on fruit flies according to
methods A, B and D.

| | | | | | |
|---|---|---|---|---|---|
| Com- | | Confidence Intervals | No. of | | |
| pound | LD₅₀ | at 95% for LD₅₀ | Organisms | | |
| No. | (μg/insect) | (μg/insect) | Tested | Slope | g |

Method A

| | | | | | |
|---|---|---|---|---|---|
| 1 | 0.00002 | 0.00000-0.00011 | 124 | 0.500 ± | 0.238 |
| | | | | 0.124 | |
| 2 | 2.27 × 10⁻⁶ | 0.00000-0.00002 | 150 | 0.308 ± | 0.175 |
| | | | | 0.066 | |
| 3 | 5.02 × 10⁻⁶ | 0.00000-0.00008 | 114 | 0.340 ± | 0.362 |
| | | | | 0.104 | |
| 4 | 0.00012 | n.d. | 114 | 0.385 ± | 0.860 |
| | | | | 0.109 | |
| 5 | 0.00016 | 0.00000-0.00226 | 114 | 0.296 ± | 0.401 |
| | | | | 0.096 | |
| 6 | 0.00015 | 0.00000-0.00092 | 114 | 0.696 ± | 0.484 |
| | | | | 0.155 | |
| 7 | 0.00020 | 0.00000-0.00154 | 114 | 0.395 ± | 0.527 |
| | | (90%) | | 0.104 | |
| 8 | 0.00037 | n.d. | | 0.453 ± | 0.967 |
| | | | | 0.115 | |
| 9 | 0.00003 | 0.00000-0.00043 | 114 | 0.326 ± | 0.405 |
| | | | | 0.106 | |
| Imida- | 0.01253 | 0.00523-0.01637 | 75 | 2.218 ± | 0.648 |
| cloprid | | | | 0.911 | |

Method B

| | | | | | |
|---|---|---|---|---|---|
| 2 | 0.00398 | 0.00000-0.2415 | 48 | 0.484 ± | 0.693 |
| | | (90%) | | 0.206 | |

Method D

| | | | | | |
|---|---|---|---|---|---|
| 5 | 4.74974 | 3.21963-20.59868 | 75 | 1.602 ± | 0.681 |
| | | (90%) | | 0.674 | |
| 7 | 0.96796 | 0.72391-2.06494 | 75 | 2.624 ± | 0.366 |
| | | | | 0.810 | |

n.d.—Data not available

The fruit fly larvae are less sensitive to the toxins than adult flies.

The values of LD₅₀ obtained for the compounds of Examples 5 and 8 are much higher than those obtained for the adult fly, therefore these compounds are less toxic for larvae than for adult flies.

### Toxicity to House Fly M. domestica

Toxicity parameters for adult house flies, treated topically with compounds of Examples 5 and 8, are given in Table 2.

Although these insects are approximately 8 fold larger than fruit flies, analysis of said table allows to conclude that the compounds tested are much less toxic (2 to 3 orders of magnitude) for this type of flies than for fruit flies.

LD₅₀ values correspond only to a moderate insecticidal activity.

TABLE 2
Toxicity parameters of compounds tested on adult house flies

| | | | | | |
|---|---|---|---|---|---|
| Com- | | Confidence Intervals | No. of | | |
| pound | LD₅₀ | at 95% for LD₅₀ | Organisms | | |
| No. | (μg/insect) | (μg/insect) | Tested | Slope | g |

Method E

| | | | | | |
|---|---|---|---|---|---|
| 5 | 1.06481 | n.d. | 40 | 0.228 ± | 1.561 |
| | | | | 0.146 | |
| 8 | 0.64404 | n.d. | 30 | 0.383 ± | 0.863 |
| | | | | 0.182 | |

### Toxicity to Brine Shrimp

Toxicity parameters for assays in which brine shrimp larvae are to the compounds in saline solution are given in Table 3. Data obtained od correlation (g<0.4) and high LC₅₀ values, indicating a low toxicity type of organisms.

TABLE 3
Toxicity parameters of compounds tested on brine shrimp larvae,
method F.

| | | | | | |
|---|---|---|---|---|---|
| Com- | | Confidence Intervals | No. of | | |
| pound | LC₅₀ | at 95% for LC₅₀ | Organisms | | |
| No. | (μg/mL) | (μg/mL) | Tested | Slope | g |

Method F

| | | | | | |
|---|---|---|---|---|---|
| 1 | 100.62 | 90.03-125.27 | 50 | 8.862 ± | 0.342 |
| | | | | 2.644 | |
| 2 | 64.30 | 57.75-77.28 | 50 | 9.217 ± | 0.315 |
| | | | | 2.638 | |
| 3 | 144.71 | 128.12-172.71 | 50 | 7.763 ± | 0.303 |
| | | | | 2.182 | |
| 4 | 358.92 | 324.89-400.42 | 60 | 9.770 ± | 0.170 |
| | | | | 2.057 | |
| 5 | 38.36 | 8.244-3258.70 | 60 | 0.423 ± | 0.655 |
| | | (90%) | | 0.175 | |
| 6 | 125.48 | 113.08-139.97 | 50 | 10.275 ± | 0.213 |
| | | | | 2.421 | |
| 7 | 261.04 | 220.59-320.09 | 50 | 8.601 ± | 0.778 |
| | | (90%) | | 2.195 | |
| 8 | 220.41 | 199.24-249.71 | 50 | 10.359 ± | 0.253 |
| | | | | 2.659 | |
| 9 | 671.50 | 567.31-807.72 | 60 | 8.734 ± | 0.591 |
| | | (90%) | | 1.862 | |
| Imida- | 0.03121 | 0.02387-0.04593 | 100 | 2.435 ± | 0.417 |
| cloprid | | | | 0.802 | |

### Insecticidal Effect on Adult White Fly

The results of the bioassays of the insecticidal compounds on adult whiteflies T. vaporariorum are given in Table 4. Five compounds were tested, which were applied spraying 600 μL of each compound solution (prepared according to method G) on the leaves of tomato plants infested with a known number of adult whitefly. After 14 hours, the number of dead insects (or eventually of insects that disappeared) was counted. These assays were performed at room temperature, between 20 and 25° C.

From the data in Table 4, it is found that the compounds of Examples 4 and 9 show efficacy as insecticides.

TABLE 4
Insecticidal effect againts adult white fly,
assayed by spraying infested tomato leaves.

| Compound No. | Concentration (μg/mL) | % Control of White Fly |
|---|---|---|
| 3 | 1.3 | 0 |
| 4 | 2.5 | 50 |
| 6 | 1.3 | 0 |
| 7 | 4.8 | 0 |
| 9 | 2.5 | 85 |

### Discussion and Conclusions

Bioassays were performed using a range of compounds and treatment techniques in different species of arthropods. Adult fruit flies, treated topically, showed high levels of sensitivity to the compounds tested, such that the LD₅₀ values determined are much lower than that for the reference insecticide “imidacloprid”. However some variation was observed in the toxicity effect produced by the different compounds.

The slope of the log-probit regression line was generally small and much smaller than that for imidacloprid. When treated by incorporation into the adult diet the compounds were much less toxic. Topical treatment of the larval stage was also much less toxic but the regression line slope increased uniformly.

Some of the compounds were tested by topical application on adult house fly and were much less toxic compared to the fruit fly adults.

The slopes of the log-probit regression line were similar to those obtained by the same type of treatment used in adult fruit fly, suggesting a mechanism of action similar although with less activity and some selectivity.

Contrary to the high insectividal activity found, the compounds have a very low toxicity against brine shrimps.

The high values of LC₅₀ are associated with steep regression lines.

It can be concluded that these compounds show a very low toxicity towards this type of organisms in saline medium, not producing toxicity in these ecosystems.

In the test of spraying the compounds on leaves infested with adult white flies the compounds of Examples 4 and 9 were found to be promising for activity against the white fly. These compounds also showed high toxicity against adult fruit flies.

### REFERENCES

1. Haynes, L. J.; Plimmer, J. R Q. Rev. Chem. Soc. 1960, 14, 292-315.
2. Devon, T. K.; Scott, A. I. Handbook of Naturally Occurring Compounds; Academic Press: New York, 1972; Vol. II, pp. 79-175 (quoted in Ref. 8).
3. Marshall, P. G. In Chemistry of Carbon Compounds; Rodd, E. H., Ed.; Elsevier: New York, 1970; Vol. II D, Chapter 17 (quoted in Ref. 8).
4. (a) Schmitz, F. J.; Kraus, K. W.; Ciereszko, L. S.; Sifford, D. H.; Weinheimer, A. J. Tetrahedron Lett. 1966, 7, 97-104; (b) Cimino, G.; De Stefano, S.; Minale, L.; Fattorusso, E. Tetrahedron 1972, 28, 333-341; (c) Cafieri, F.; Fattorusso, E.; Santacroce, C.; Minale, L.; Tetrahedron 1972, 28, 1579-1583; (d) Faulkner, D. J. Tetrahedron Lett. 1973, 14, 3821-3822; (e) Rothberg, I.; Shubiak, P. Tetrahedron Lett. 1975, 16, 769-722; (f) Cimino, G.; De Stefano, S.; Guerriero, A.; Minale, L. Tetrahedron Lett. 1975, 16, 1417-1420.
5. Ma, S.; Schi, Z.; Yu, Z. Tetrahedron 1999, 55, 12137-12148.
6. Larock, R. C.; Riefling, B.; Fellows, C. A. J. Org. Chem. 1978, 43, 131-137.
7. Brownbridge, P.; Chan, T. H. Tetrahedron Lett. 1980, 21, 3431-3434.
8. Cardellach, J.; Estopa, C.; Font, J.; Moreno-Mañas, M.; Ortuño, R. M.; Sachez-Ferrando, F.; Valle, S.; Vilamajo, L. Tetrahedron 1982, 38, 2377-2394.
9. Kotora, M.; Negishi, E. Synthesis 1997, 121-128.
10. Klein Gebbinck, E. A.; Stork, G. A.; Jansen, B. J. M.; of Groot, A. Tetrahedron 1999, 55, 11077-11094.
11. Choudhury, P. K.; Foubelo, F.; Yus, M. Tetrahedron 1999, 55, 10779-10788.
12. Figueredo, M.; Font, J.; Virgili, A. Tetrahedron 1987, 43, 1881-1886.
13. Rauter, A. P.; Ferreira, M. J.; Font, J.; Virgili, A.; Figueredo, M.; Figueiredo, J. A.; Ismael, M. I.; Canda, T. L. J. Carbohydr. Chem. 1995, 14, 929-948.
14. Rauter, A. P.; Figueiredo, J. A.; Ismael, M. I.; Pais, M. S.; Gonzalez, A. G.; Dias, J.; Barrera, J. B. J. Carbohydr. Chem. 1987, 6, 259-272.
15. Rauter, A. P.; Figueiredo, J.; Ismael, M.; Canda, T. L.; Font, J.; Figueredo, M. Tetrahedron: Asymmetry 2001, 12, 1131-1146.
16. Csuk, R.; Furstner, A.; Weidmann, H. J. Chem. Soc., Chem. Commun. 1986, 775.
17. Csuk, R.; Glanzer, B. I.; Hu, Z.; Boese, R. Tetrahedron 1994, 50, 1111-1124.
18. Hanessian, S.; Girard, C. Synlett 1994, 10, 865-867.
19. Rao, A. S. Tetrahedron 1983, 39, 2323-2367.
20. Garem, B. Tetrahedron 1978, 34, 3353-3383.
21. Rodrigues, J.; Dulcere, J. P. Synthesis, 1993, 1177-1202.
22. Waggins, L. F. Nature, 1950, 165.
23. Ohle, M.; Vargha, L. V. J. Chem. Soc. 1959, 2717.
24. Mitsunobu, O. Synthesis 1981, 1.
25. Szeja, W. Synthesis 1985, 983-985.
26. Kwart, H.; Hoffman, D. M. J. Org. Chem. 1966, 31, 419-425.
27. Gutshe, C. D. Organic Reactions, 1954, 8, 364.
28. Rauter, A. P.; Figueiredo, J. A.; Ismael, M. I., Carbohydr. Res. 1989, 188, 19-24.
29. Dax, K.; Rauter, A. P.; Stütz, A. E.; Weidmann, H., Liebigs Ann. Chem. 1981, 1768-1773.
30. Robertson, J. L.; Preisher, H., Pesticide Bioassays with Arthropods, 1992, CRC Press, Boca Raton, Fla.
31. Finney, D. J., Probit Analysis, 1972, 3^{rd} Ed., Cambridge University Press, London, p. 79.

1. Pesticidal compound of formula (I) 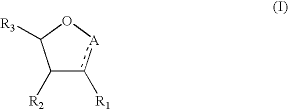
wherein [symbol]
represents a carbon-carbon single or double bond; [symbol]
represents —CH(R₄)—, if said carbon-carbon bond is a single bond,
wherein
R₄ represents, independently, hydrogen, alkoxy or substituted alkoxy, or
R₁ and R₄, taken together, represent an alkylidenedioxy or a substituted alkylidenedioxy group; or [symbol]
represents ═C(R₅)—C(═O)—, if said carbon-carbon bond is a double bond,
wherein:
R₅ represents hydrogen or halogen;
R₁ and R₂ represent, independently, hydrogen, halogen, alkoxy, substituted alkoxy or an ester group; or
R₁ and R₂, together with the carbon atoms to which they are attached, represent an oxirane ring; or
R₁ and R₂, taken together, represent an akylidenedioxy or substituted alkylidenedioxy group; and
R₃ represents —CH₂R₆,
wherein
R₆ represents an ester group,
oxiranyl,
or a group of formula 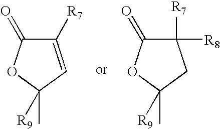
wherein
R₇ represents hydrogen or alkyl,
R₈ represents phenylsulfanyl, phenylselenyl, phenylsulfoxy or phenylselenoxy, and
R₉ represents hydrogen, ethoxycarbonyl or carbamoyl.

2. Pesticidal compound of formula (I) according to claim 1, wherein [symbol]
represents a carbon-carbon single bond; [symbol]
represents —CH(R₄)—,
wherein
R₄ represents, independently, hydrogen, alkoxy or substituted alkoxy, or
R₁ and R₄, taken together, represent an alkylidenedioxy or substituted alkylidenedioxy group;
R₁ and R₂ represent, independently, hydrogen, alkoxy, substituted alkoxy or an ester group, or
R₁ and R₂, together with the carbon atoms to which they are attached, represent an oxirane ring; or
R₁ and R₂, taken together, represent an alkylidenedioxy or a substituted alkylidenedioxy group; and
R₃ represents a group of formula 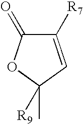
wherein
R₇ represents hydrogen or alkyl, and
R₉ represents hydrogen, ethoxycarbonyl or carbamoyl.

3. Pesticidal compound of formula (I) according to claim 2 having formula (IA) 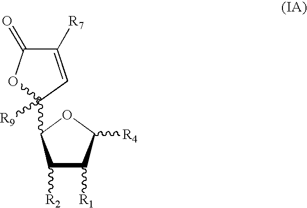
wherein R₁, R₂, R₄, R₇ and R₉ are as defined in claim 2.

4. Compound according to claim 3, wherein R₁ and R₄, taken together, represent isopropylidenedioxy group, R₂ represents benzyloxy, R₇ represents methyl and R₉ represents hydrogen.

5. Compound according to claim 4, which is a D-gluco derivative, i.e. 3-O-benzyl-6,7-dideoxy-1,2-O-isopropylidene-7-methyl-α-D-gluco-oct-6-enefuranurono-8,5-lactone.

6. Compound according to claim 4, which is a D-alo derivative, i.e. 3-O-benzyl-6,7-dideoxy-1,2-O-isopropylidene-7-methyl-α-D-alo-oct-6-enefuranurono-8,5-lactone.

7. Compound according to claim 3, wherein R₁ and R₄, taken together, represent an isopropylidenedioxy group, R₂ represents benzyloxy and R₇ and R₉ represents hydrogen.

8. Compound according to claim 7, which is a D-alo derivative, i.e. 3-O-benzyl-6,7-dideoxy-1,2-O-isopropylidene-α-D-alo-oct-6-enefuranurono-8,5-lactone.

9. Compound according to claim 7, which is a D-gluco derivative, i.e. a 3-O-benzyl-6,7-dideoxy-1,2-O-isopropylidene-α-D-gluco-oct-6-enefuranurono-8,5-lactone.

10. Pesticidal compound of formula (I) according to claim 1, wherein [symbol]
represents a carbon-carbon double bond; [symbol]
represents ═C(R₅)—C(═O)—,
wherein,
R₅ represents hydrogen or halogen;
R₁ represents hydrogen or halogen; and
R₃ represents —CH₂R₆,
wherein
R₆ represents an ester group.

11. Pesticidal compound of formula (I) according to claim 10, which has formula (IB) 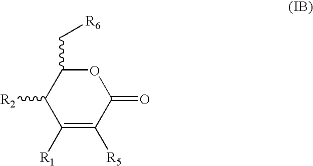
wherein R₁, R₂, R₅ and R₆ are as defined in claim 10.

12. Compound according to claim 11, wherein R₁ represents hydrogen, R₂ represents —OC(═O)CH₃, R₅ represents Br and R₆ represents —OC(═O)CH₃.

13. Compound according to claim 12, which a D-erythro derivative, i.e. 4,6-di-O-acetyl-2-bromo-2,3-dideoxy-D-erythro-hex-2-ene-1,5-lactone.

14. Pesticidal compound of formula (I) according to claim 1, wherein [symbol]
represents a carbon-carbon single bond; [symbol]
represents —CH(R₄)—,
wherein
R₄ represents, independently, hydrogen, alkoxy or substituted alkoxy, or
R₁ and R₄, taken together, represent an alkylidenedioxy or substituted alkylidenedioxy group;
R₁ and R₂ represent, independently, hydrogen, alkoxy or substituted alkoxy, or
R₁ and R₂, together with the carbon atoms to which they are attached, represent an oxirane ring; or
R₁ and R₂, taken together, represent an alkylidenedioxy or substituted alkylidenedioxy group; and
R₃ represents a group of formula 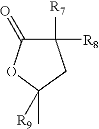
wherein
R₇ represents hydrogen or alkyl,
R₈ represents phenylsulfanyl, phenylselenyl, phenylsulfoxy or phenylselenoxy, and
R₉ represents hydrogen, ethoxycarbonyl or carbamoyl.

15. Pesticidal compound of formula (I) according to claim 14 having formula (IC) 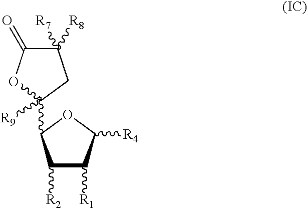
wherein R₁, R₂, R₄, R₇, R₈ and R₉ are as defined in claim 14.

16. Pesticidal compound of formula (I) according to claim 15, wherein R₁ and R₂, taken together with the carbon atoms to which they are attached, form an oxirane ring, R₄ represents methoxy, R₇ represents methyl, R₈ represents phenylselenyl and R₉ represents hydrogen.

17. Compound according to claim 16, which is a 2,3-anhydro-β-L-gulo derivative, i.e. methyl (7R)- and methyl (7S)-2,3-anhydro-6,7-dideoxy-7-methyl-7-phenylselenyl-β-L-gulo-octofuranurono-8,5-lactone.

18. Pesticidal compound of formula (I) according to claim 15, wherein R₁ and R₄, taken together, form an isopropylidenedioxy group, R₂ represents hydrogen, R₇ represents methyl, R₈ represents phenylselenyl and R₉ represents hydrogen.

19. Compound according to claim 18, which is a D-ribo derivative, i.e. (7R)- and (7S)-3,6,7-trideoxy-1,2-O-isopropylidene-7-methyl-7-phenylselenyl-α-D-ribo-octofuranurono-8,5-lactone.

20. Pesticidal compound of formula (I) according to claim 15, wherein R₁ and R₄, taken together, form an isopropylidenedioxy group, R₂ represents benzyloxy, R₇ represents methyl, R₈ represents phenylselenyl and R₉ represents hydrogen.

21. Compound according to claim 20, which is a D-gluco derivative, i.e. a (7R)- and (7S)-3-O-benzyl-6,7-dideoxy-1,2-O-isopropylidene-7-methyl-7-phenylselenyl-α-D-gluco-octofuranurono-8,5-lactone.

22. Pesticidal compound of formula (I) according to claim 1, wherein [symbol]
represents a carbon-carbon single bond; [symbol]
represents —CH(R₄)—,
wherein
R₄ represents alkoxy or substituted alkoxy;
R₁ and R₂, taken together with the carbon atoms to which they are attached, represent an oxirane ring; and
R₃ represents oxiranyl.

23. Pesticidal compound of formula (I) according to claim 22 having formula (ID) 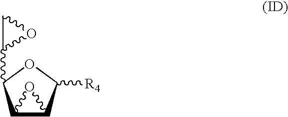
wherein R₄ is as defined in claim 22.

24. Pesticidal compound of formula (I) according to claim 23, wherein R₄ represents methoxy.

25. Compound according to claim 24, which is 2,3; 5,6-dianhydro-α-L-gulo derivative, i.e. methyl 2,3; 5,6-dianhydro-β-L-gulofuranoside.

26. Pesticidal compound of formula (I) according to claim 1, wherein [symbol]
represents a carbon-carbon single bond; [symbol]
represents —CH(R₄)—,
wherein
R₄ represents, independently, hydrogen, alkoxy or substituted alkoxy, or
R₁ and R₄, taken together, represent an alkylidenedioxy or substituted alkylidenedioxy group; or
R₁ and R₂ represent, independently, hydrogen, alkoxy or substituted alkoxy, or
R₁ and R₂, taken together, represent an alkylidenedioxy or substituted alkylidenedioxy group; and
R₃ represents oxiranyl.

27. Pesticidal compound of formula (I) according to claim 26 having formula (IE) 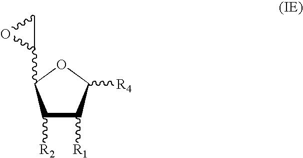
wherein R₁, R₂ and R₄ are as defined in claim 26.

28. Process for the preparation of a compound of Formula I of any of claims 1 to 27, wherein:
a) for the preparation of compounds of formula (IA), a lactone of formula (IC′) [compound of formula (IC), wherein R₁ and R₄ represent, taken together, isopropylidenedioxy, R₂ represents benzyloxy, R₇ represents hydrogen or methyl, R₈ represents XPh, wherein X represents S or Se, and R₉ represents hydrogen] is converted into a butenolide of formula (IA′) [compound of formula (IA), wherein R₁ and R₄ represent, taken together, isopropylidenedioxy, R₂ represents benzyloxy, R₇ represents hydrogen or methyl and R₉ represents hydrogen], according to the following scheme: 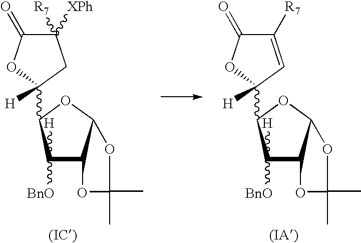
b) for the preparation of compounds of formula (IB), a compound of formula (II) is converted into an α,β-unsaturated hexono-1,5-lactone of formula (IB′) [compound of formula (IB), wherein R₁ represents hydrogen, R₂ and R₆ represent acetoxy and R₅ represents bromine], according to the following scheme: 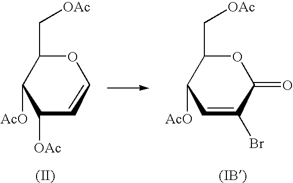
c) for the preparation of compounds of formula (IC) a precursor diepoxide of formula (ID′) [compound of formula (ID), wherein R₁ and R₂ represent, taken together, oxyranyl and R₄ represents methoxy] is converted into a lactone of formula (IC″) [compound of formula (IC), wherein R₁ and R₂ represent, taken together, oxyranyl, R₄ represents methoxy, R₇ represents hydrogen or methyl, R₈ represents XPh, wherein X represents S or Se, and R₉ represents hydrogen], according to the following scheme: 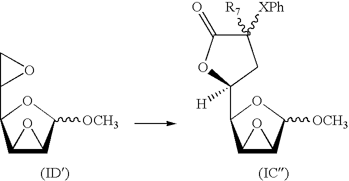
or
the epoxide precursor of formula (IE′) [compound of formula (IE), wherein R₁ and R₄ represent, taken together, isopropylidenedioxy and R₂ represents benzyloxy or hydrogen] is converted into a lactone of formula (IC′″) [compound of formula (IC), wherein R₁ and R₄ represent, taken together, isopropylidenedioxy, R₂ represents benzyloxy or hydrogen, R₇ represents hydrogen or methyl, R₈ represents XPh, wherein X represents S or Se, and R₉ represents hydrogen], according to the following scheme: 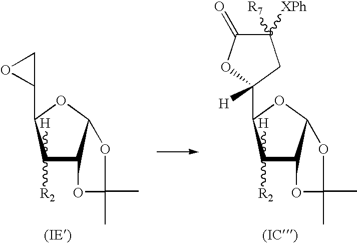
d) for the preparation of compounds of formula (ID) a compound of formula (III) is converted into a diepoxide of formula (ID″) [compound of formula (ID), wherein R₄ represents methoxy], according to the following scheme: 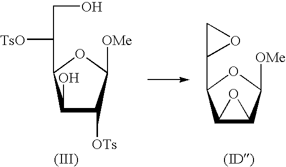
e) for the preparation of compounds of formula (IE), a compound of formula (IV) is converted into an epoxide of formula (IE″) [compound of formula (IE), wherein R₁ and R₄ represent, taken together, isopropylidenedioxy and R₂ represents benzyloxy], according to the following scheme:

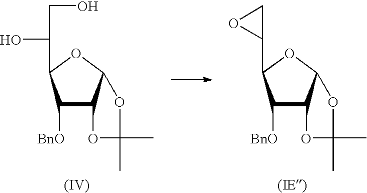

29. Use of a compound of formula (I) of any of the claims 1 to 27 as a pesticide.

30. The use of claim 29, wherein the pests are arthropods.

31. The use of claim 30, wherein the arthropods are insects.

32. The use of claim 31, wherein the insects are fruit fly (Drosophila melanogaster), house fly (Musca domestica) and white fly (Trialeurodes vaporarium).

33. A method for controlling pests, comprising the application of an effective amount of compounds of formula (I) to said pests or their locus.

34. The method of claim 33, wherein the pests are arthropods.

35. The method of claim 34, wherein the arthropods are insects.

36. The method of claim 35, wherein the insects are fruit fly (Drosophila melanogaster), house fly (Musca domestica) and white fly (Trialeurodes vaporarium).
